(19) United States
(12) Patent Application Publication
(10) Pub. No.: US 2018/0243108 A1
(43) Pub. Date: Aug. 30, 2018

(54) INTER-BODY SPACER

(71) Applicant: ORTHOSPINE GLOBAL LLC, Wilmington, DE (US)

(72) Inventors: Rudi VERBEEK, Kempton Park (ZA); Brendan O'TOOLE, Pretoria (ZA)

(21) Appl. No.: 15/558,892

(22) PCT Filed: Jan. 11, 2016
(86) PCT No.: PCT/ZA2016/050001

§ 371 Date: Sep. 15, 2017

Related U.S. Application Data
(60) Provisional application No. 62/114,742, filed on Feb. 11, 2015.

(51) Int. Cl. A61F 2/44; A61F 2/46

(52) U.S. Cl. CPC A61F 2/4425; A61F 2002/30405; A61F 2/4611

(57) ABSTRACT

The invention provides an inter-body spacer in the form of a vertebral body replacement. The spacer comprises a connector having, axially aligned, a socket and threaded socket. A threaded screw is receivable in the threaded socket of the connector at one side of the screw and an endplate receiving head is provided on the other end of the screw for receiving an endplate perpendicular to the axis. The spacer further comprises a cylindrical extension with one end rotatably receivable in the socket of the connector and an endplate receiving head on the other end of the extension for receiving an endplate perpendicular to the axis. A locating formation is provided on each endplate receiving head for locating the endplate at a specific angle about the axis. The spacer further comprises a selection of one or more end plates receivable on the endplate receiving head with the tissue engaging surface of the endplates being transverse to the axis of the spacer at differing angles. The invention also extends to a elongate thoracolumbar lateral plate.

### TECHNICAL FIELD OF THE INVENTION

This invention relates to an Inter-body Spacer, more specifically it relates to a Vertebral Body Replacement (VBR) implant.

### BACKGROUND TO THE INVENTION

Inter-body spacers are used to replace the, or a part of a vertebral body and its associated upper and lower intervertebral discs. Typically a bone graft with or without a metal plate is required to reconstruct the spine and to provide stability. This type of surgery is mostly carried out on patients with a spinal fracture, tumour, or infection which causes compression of the spinal nerves.

It is an object of the invention to provide an inter-body spacer and a VBR introducer, or implantation tool, with improved ease of implantation.

### GENERAL DESCRIPTION OF THE INVENTION

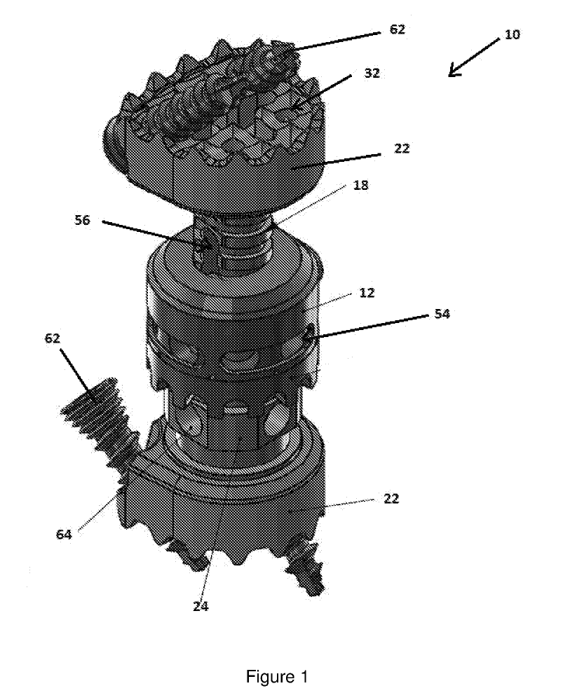

In this description of the invention, reference to an inter-body spacer refers to an inter-body spacer, by the applicant as described in South African patent application number 2014/3891, in the form of a vertebral body replacement as shown in FIG. 1, which spacer comprises:
a connector having, axially aligned, a socket and threaded socket;
a threaded screw receivable in the threaded socket of the connector at one side of the screw and an endplate receiving head on the other end of the screw for receiving an endplate perpendicular to the axis;
a cylindrical extension with one end rotatably receivable in the socket of the connector and an endplate receiving head on the other end of the extension for receiving an endplate perpendicular to the axis;

a locating formation on each endplate receiving head for locating the endplate at a specific angle about the axis; and
a selection of one or more end plates receivable on the endplate receiving head with the tissue engaging surface of the endplates being transverse to the axis of the spacer at differing angles. An inter-body spacer introducer or implantation tool is clamped onto the inter-body spacer to introduce the spacer between vertebrae during surgery.

According to a first aspect of the invention, an inter-body spacer wherein the locating formation on each endplate receiving head for locating the endplate at a specific angle about the axis is one or more notches formed in the head or endplate with a complementary stub formations which fits snugly into the notches formed on either the endplate or head, as the case may be. In a preferred embodiment of the invention, a pair of notches is formed in the head and the complementary stub.

The applicant found that the use of the notch and stub formation, especially in combination with the Morse taper significantly improves the time required and ease of fitting the endplate to the head during surgery. The surgeon can feel a single reassuring and positive mating of the formations. The notch and stub formations are also provided with the morse taper to improve the fit.

In addition, the surgeon can select, during the surgical procedure the correct size and lordotic angle required for a lordosis directional benefit, which can be fitted to the heads at the correct orientation.

The cylindrical extension may preferably be provided with one or more lateral threaded sockets extending into the connector transverse or perpendicular to the axis. The invention provides an introducer having a thread tipped rod which engages, in use, one of the lateral threaded sockets.

The end plates may be circular in shape and will also incorporate anatomically shaped end plates for improved load bearing capacity. Standalone end plates will also form part of the design in which correctly angulated screw holes are present in the end plate thereby permitting screw fixation to the above and below vertebral bodies, typically for cervical use. The surgeon can during the surgical procedure decide, if necessary, to fit and fix one or both endplates with angulated screws.

End plates may be coated with Hydroxylapatite for improved bony ingrowth.

The thread is preferably square thread.

The endplate receiving heads and the end plates may be provided with complementary Morse* taper formations to attach the end plates. The Morse Taper (2 degrees and 55 minutes) provides a grip and hold feature between the end plate and its end plate receiving head.

The cylindrical extension may be hollow and the treaded screw may fit into the hollow with the screw and cylinder having complementary formations to prevent rotation relative to each other. It will be appreciated that this ensures that with the screw inserted into the cylindrical extension that the orientation the location formations on the opposed receiving heads remain fixed relative to each while the length of the spacer is being adjusted.

The connector may be in the form of or include an axially fixed pinion rotatable about the connector with pinion gears at one end of the pinion facing towards the lateral threaded sockets. The other end of the pinion engages the threads of the threaded screw and by turning the pinion relative to the connector, screw and cylindrical extension, the length of the inter-body spacer can be adjusted.

The introducer of which the rod engages, in use, one of the lateral threaded sockets may also include a sleeve over the rod, which rod and sleeve can be rotated relative to each other and which sleeve at the threaded end of the rod is provided with gears for engaging, in use, the gears of the pinion to rotate the pinion, in turn, to adjust the length of the inter-body spacer.

The pinion may also be provided with one, preferably more, threaded holes perpendicular to the axis of the inter-body spacer through which the pinion can be rotationally fixed to the connector with a grub screw once the length of the inter-body spacer has been adjusted as required.

A sleeve or grub screw retaining ring may preferably be provided to retain the grub screws. A slot provided in the retaining ring may allow access for a tool to engage the grub screws.

The cylindrical extension is provided with a circular slot about the cylinder proximate the end receivable in the connector. A spring loaded lock ring is provided to fit into the slot and a slot provided inside the pinion to prevent the cylindrical extension to move axially relative to the pinion.

A grub screw can be fitted into a threaded hole provided in the connector to fix the connector to the cylindrical connection.

The invention also extends to an inter-body spacer introducer or implantation tool, which introducer comprises:
a thread tipped rod which engages, in use, one of the lateral threaded sockets of the connector;
a sleeve over the rod, which rod and sleeve can be rotated relative to each other and which sleeve at the threaded end of the rod is provided with gears for engaging, in use, the gears of the pinion to rotate the pinion, in turn, to adjust the length of the inter-body spacer;
an outer tube covering the rod and sleeve with a handle at the one end and locating means for locating the inter-body spacer at the other end with the thread tipped rod engaged as well as the gears of the sleeve engaged, with the locating means being in the form of U-shaped arms which fit snugly and complementally over the connector to locate the inter-body spacer and the introducer square to each other; and wherein
the end of the sleeve opposite the gears extends past the handle of the outer tube and fitted with its own handle; and wherein
a window is provided in the sleeve, between the handle of the outer tube and the handle of the sleeve to provide access to rod, which rod is provided with gripping means which can be gripped through the window to allow the rod to be rotated relative to the sleeve and outer tube.

In use a person will fit opposed end plates to the inter-body spacer at the correct lordotic angle and at the correct orientation to each other. The person will then fit the inter-body spacer between the U-shaped arms of the introducer and screw the threaded tip of the rod into a threaded hole provided in the connector until it is snugly and securely engaged and with the gears of the sleeve engaging the gears of the pinion. The inter-body spacer can then be introduced between vertebrae and its length adjusted to any length, at infinitely small adjustments, as required. After adjustment all moving parts are locked into position.

The invention also extends to a kit for implanting an inter-body spacer, which kit comprises an inter-body spacer as described above and an introducer as described above.

It will be appreciated that the vertebral body replacement is axially adjustable, modular and that a surgeon can choose different end plates with regard to its footprint and lordotic angles. It will further be appreciated that the vertebral body replacement according to the invention limits the possibility of making a mistake with regard to the angles despite its modular design by means of the complemental notches provided in the heads and it associated end plates. In addition, the use of the complementary notch and stub formations on the head and endplate lessens the time required for a surgeon to fit the head in the correct orientation and provides reassuring feedback to the surgeon that the orientation is correct.

The invention also extends to an elongate thoracolumbar lateral plate, which can be used to complement the inter body spacer as described above. The lateral plate comprises two parts of which one has a slot and the other a lockable screw. The screw is receivable in the slot and the two parts are slideably connected. Once the length of the lateral plate is determined, it can, in use, be set to that length by means of the lockable screw. Complementary retaining formations provide support for lengthwise alignment, in use. The opposed end of each part is provided with a pair of first countersunk holes for receiving surgical screws there through. A second countersunk hole is provided adjacent each first countersunk hole, intersecting therewith. A notched screw is provided in each second hole with the notch allowing the surgical screw to be turned in one position and preventing the surgical screw from coming out in another position. The inner, spinal side of the lateral plate may be provided with a pair of generally transverse notches or shoulders which extends towards the spine, in use, which provides support for the opposed top and bottom ends of the inner plate of a person's remaining vertebrae.

The Lateral plate is a telescopic type thoracolumbar plate system designed to engage with the lordosis curvature of the thoracolumbar spine whilst also including optimization of a snug fit to the complex shape of the vertebral body.

The length adjustable lateral plating system is intended for use with interbody spacers such as the vertebral body replacement described above and/or meshes cage systems, laterally in relation thereto. The plate has an inner curvature which conforms to the circular shape of the vertebral body incorporating a slim design for minimal post op soft tissue interference. It has the following advantages:
The pre-contoured design of the plate increases strength and stability whilst withstanding spinal loads;
The undercut slotted design of the telescopic sliding mechanism of the plate facilitates optimal screw/vertebral body placement intra-operatively, whilst incorporating superb stability;
The stability of the vertebral body replacement structure, as described above, incorporating intra-operative compression helps provide the secure fit which affords the bone graft optimum conditions for fusion;
The Slide plating system utilizes internal dynamization and optimal compression to maximize the principles of Wolff's law in promoting fusion;
Ease of insertion and adjustability by way of one single instrument prior to securely locking the screw;
Plates available in various lengths eg., 40 mm-120 mm at approx 14 mm intervals; and
Provides support for the opposed top and bottom ends of the inner plate of a person's remaining vertebrae.

### DETAILED DESCRIPTION OF THE INVENTION

The invention is now described by way of example with reference to the accompanying drawings.

In the drawings:

FIG. 1 shows an exploded view of a typical vertebral body replacement by the applicant;

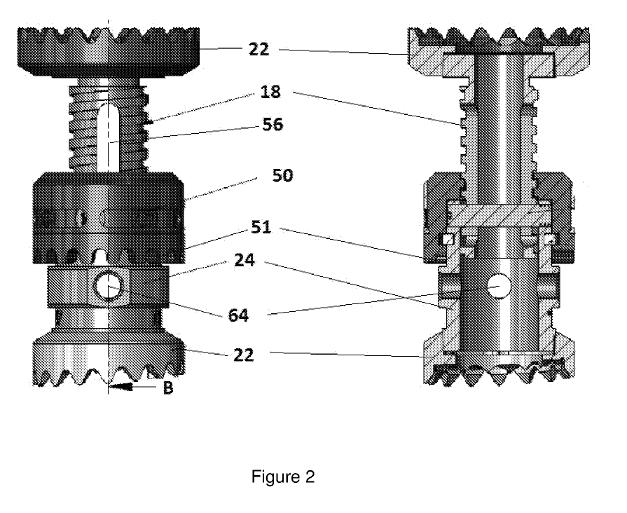

FIG. 2 shows a side and sectional side view B-B of a vertebral body replacement, in accordance with the invention;

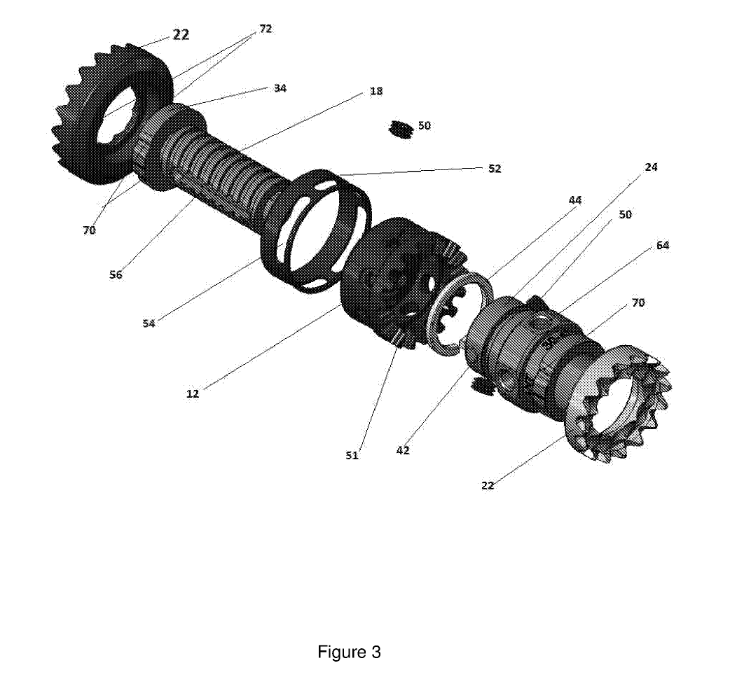

FIG. 3 shows an exploded view of the vertebral body replacement;

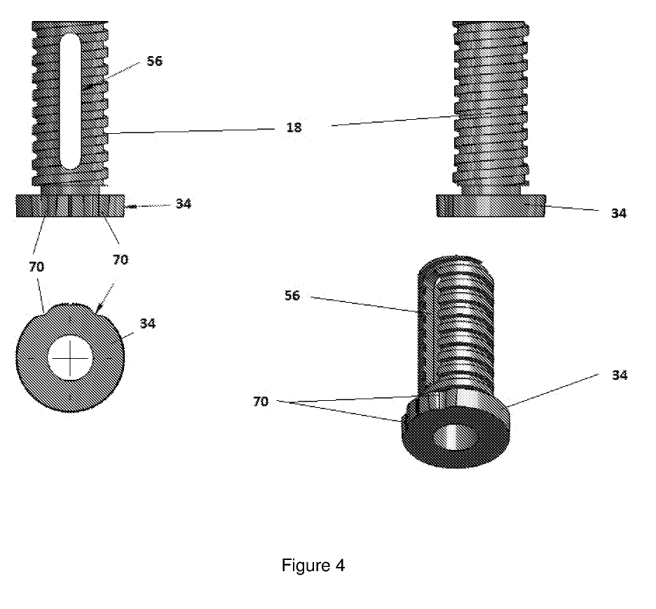

FIG. 4 shows a selection of views of a square threaded screw;

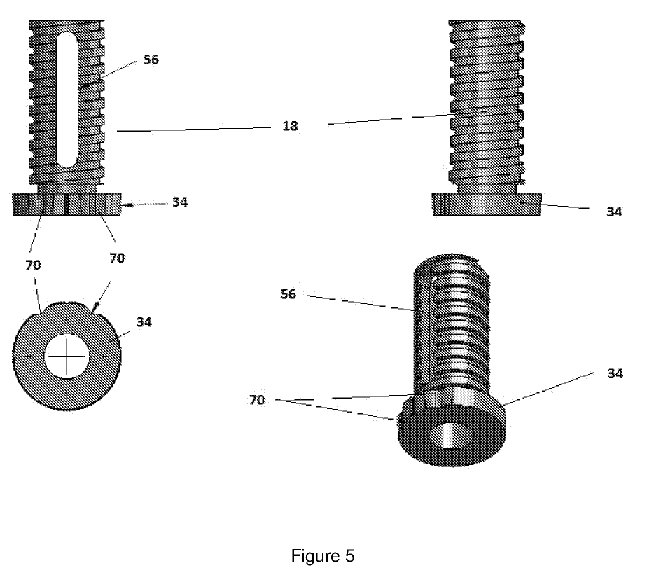

FIG. 5 shows a selection of views of an endplate;

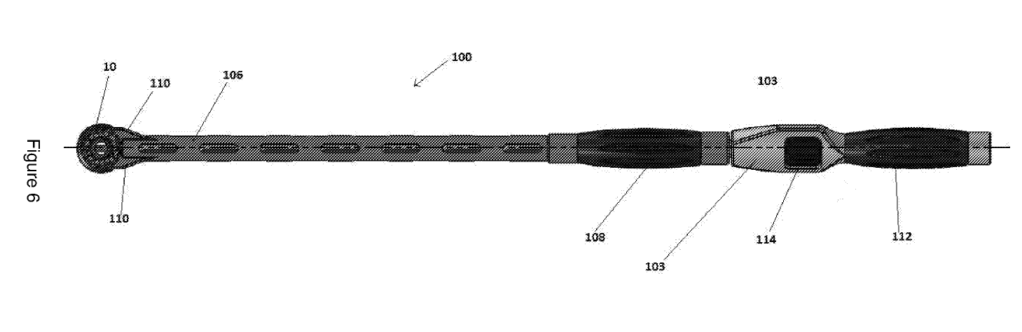

FIG. 6 shows a top view of an introducer with an engaged vertebral body replacement, in accordance with the invention;

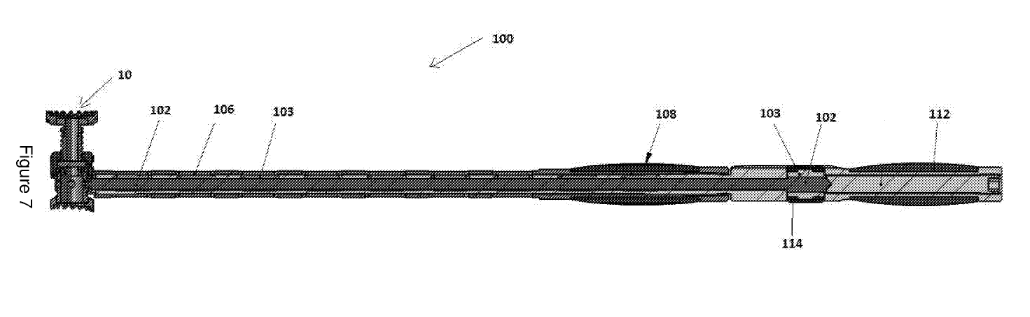

FIG. 7 shows a sectional side view through B-B of the introducer with an engaged vertebral body replacement;

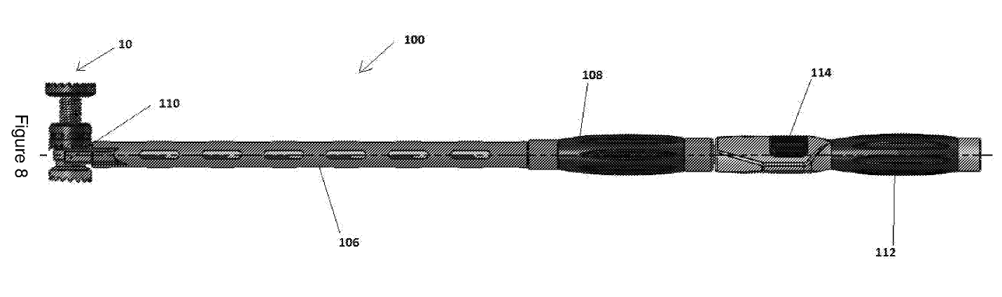

FIG. 8 shows a side view of the introducer with an engaged vertebral body replacement;

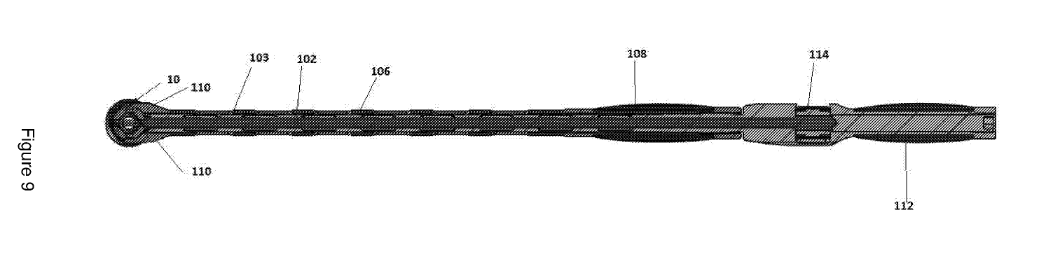

FIG. 9 shows a sectional side view through A-A of the introducer with an engaged vertebral body replacement;

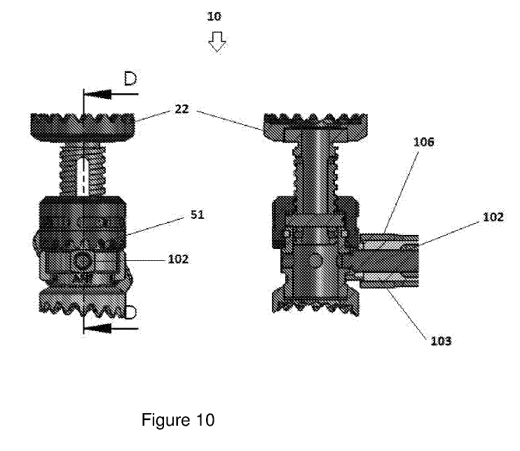

FIG. 10 shows detail of the introducer engaged with a vertebral body replacement;

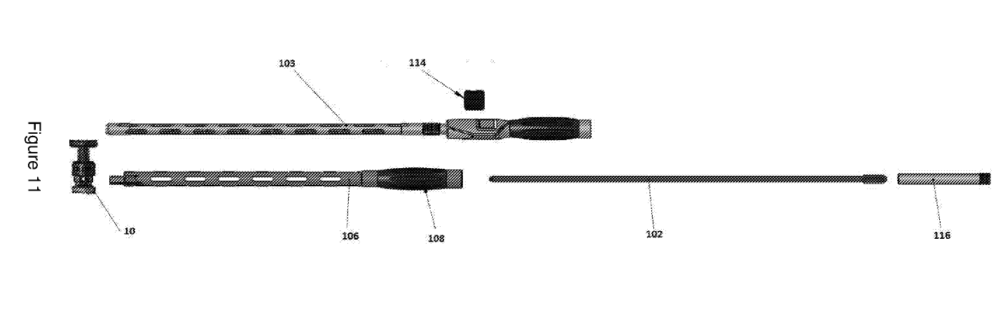

FIG. 11 shows an exploded view of the introducer engaged with a vertebral body replacement;

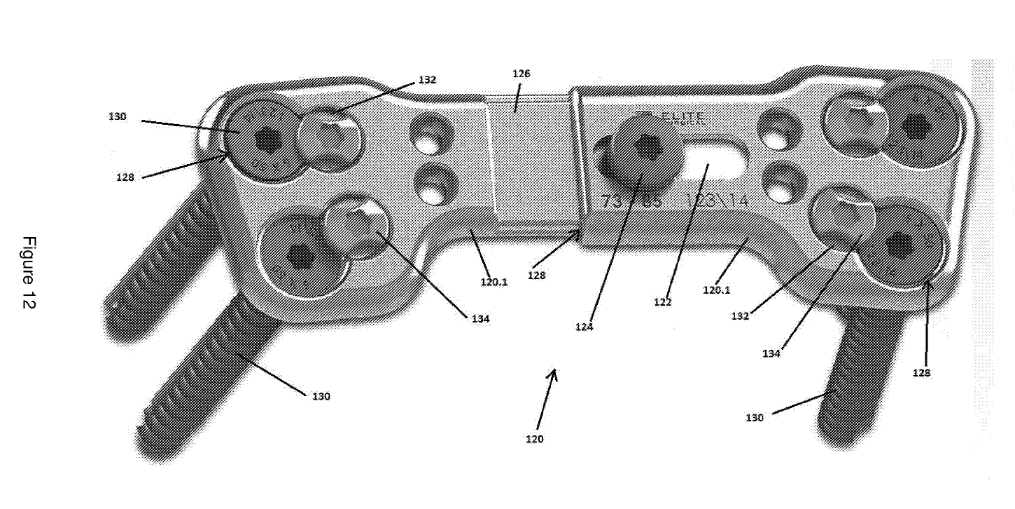

FIG. 12 shows a perspective view of and elongate thoracolumbar lateral plate, in accordance with the invention; and

FIG. 13 shows the elongate thoracolumbar lateral plate used with a mesh cage system.

Referring now to the drawings, FIG. 2 onwards, the inter-body spacer in the form of a vertebral body replacement, in accordance with the invention, is generally indicated by reference numeral 10.

The spacer 10 comprises a connector 12 having, axially aligned, a socket 14 and square threaded socket 16. The spacer 10 further comprises a square threaded screw 18 receivable in the threaded socket 16 of the connector 12 at one side of the screw and an endplate receiving head 20 on the other end of the screw for receiving an endplate 22 perpendicular to the axis. The spacer 10 further comprises a cylindrical extension 24 with one end rotatably receivable in the socket 14 of the connector 12 and an endplate receiving head 26 on the other end of the extension for receiving an endplate 22 perpendicular to the axis. A locating formation in the form of a pair of notches 70 are on each endplate receiving head 20 and 26 for locating the endplate with a complementary pair of stubs 72 at a specific angle about the axis. A selection of one or more end plates are receivable on the endplate receiving heads 20 and 26 with the tissue engaging surface 32 of the endplates being serrated and transverse to the axis of the spacer at differing angles. The endplate receiving heads 20 and 26 and the end plates 22 are provided with complementary Morse* taper formations 34 and 36 to attach the end plates to the heads. The cylindrical extension 24 is hollow and the treaded screw fits into the hollow with the screw 18 and cylinder 24 having complementary flat formations 38 and 40 to prevent rotation relative to each other. The cylindrical extension 24 is also provided with a circular slot 42 about the cylinder proximate the end receivable in the connector 12. A spring loaded lock ring 44 is provided to fit into the slot 42 and a slot 46 provided inside the connector 12. The connector 12 is further provided with a series of threaded holes 48 through the connector 12 arranged about the connector such that grub screws 50 can be screwed through the connector to engage the cylindrical extension 24 to fix the cylindrical extension and the connector and pinion 51 relative to each other. A sleeve or grub screw retaining ring 52 is provided to retain the grub screws 50. Slots 54 are provided in the retaining ring 54 may allow access for a tool to engage the grub screws. A slot 56 is provided in the threaded screw, a complementary hole 58 on the cylindrical extension 24 as well as the connector 12 to finally lock all the parts together with a sheer pin 60. The end plates 22 are provided with angled holes for receiving bone screws 62 there through.

The cylindrical extension 24 is also provided with a set of lateral threaded sockets 64 extending into the connector transverse or perpendicular to the axis for threadedly fixing and locating an introducer (FIG. 5 onwards).

A locating formation in the form of a pair of notches 70 is provided on each endplate receiving head 34 and 36 for locating the endplate at a specific angle about the axis with a complementary pair of stubs 72 formation which fits snugly into the notch is formed on the endplates 22.

Referring now to FIGS. 5 to 11, an inter-body spacer introducer or implantation tool 100 comprises a thread tipped rod 102 which engages, in use, one of the lateral threaded sockets 64 into the cylindrical extension 24 of the connector 12 and a sleeve 103 over the rod 102, which rod and sleeve can be rotated relative to each other and which sleeve at the threaded end of the rod is provided with gears 104 for engaging, in use, the gears of the pinion 51 to rotate the pinion, in turn, to adjust the length of the inter-body spacer 10. The introducer further comprises an outer tube 106 covering the rod 102 and sleeve 103 with a handle 108 at the one end and locating means 110 for locating the inter-body spacer at the other end with the thread tipped rod engaged as well as the gears of the sleeve engaged, with the locating means being in the form of U-shaped arms which fit snugly and complementally over the connector 12 to locate the inter-body spacer 10 and the introducer 100 securely and square to each other. The end of the sleeve 103 opposite the gears extends past the handle 108 of the outer tube 106 and is fitted with its own handle 112. A window 114 is provided in the sleeve 103, between the handle 108 of the outer tube 106 and the handle 112 of the sleeve 103 to provide access to the rod 102, which rod is provided with gripping means 114 which can be gripped through the window to allow the rod to be rotated relative to the sleeve and outer tube. All the components fit into each other and are held in place by means of a pin 116, which screws into the handle 112 to lock the components in place.

Referring now to FIGS. 12 and 13, which shows an elongate thoracolumbar lateral plate 120. The lateral plate 120 comprises two parts 120.1 and 120.2 of which one has a slot 122 and the other a lockable screw 124. The screw 122 is receivable in the slot and the two parts are slideably connected. Once the length of the lateral plate is determined, it can, in use, be set to that length by means of the lockable screw 124. Complementary retaining formations 126 and 128 provide support for lengthwise alignment, in use. The opposed end of each part 120.1 and 120.2 is provided with a pair of first countersunk holes 128 for receiving surgical screws 130 there through. A second countersunk hole 132 is provided adjacent each first countersunk hole 128, intersecting therewith. A notched screw 134 is provided in each second hole with the notch allowing the surgical screw to be turned in one position and preventing the surgical screw from coming out in another position. The inner, spinal side of the lateral plate 120 is provided with a pair of generally transverse shoulders (not shown) which extends towards the spine, in use, which provides support for the opposed top and bottom ends of the inner plate of a person's remaining vertebrae.

It shall be understood that the examples are provided for illustrating the invention further and to assist a person skilled in the art with understanding the invention and are not meant to be construed as unduly limiting the reasonable scope of the invention.

1. An inter-body spacer in the form of a vertebral body replacement, which spacer comprises:
a connector having, axially aligned, a socket and threaded socket;
a threaded screw receivable in the threaded socket of the connector at one side of the screw and an endplate receiving head on the other end of the screw for receiving an endplate perpendicular to the axis;
a cylindrical extension with one end rotatably receivable in the socket of the connector and an endplate receiving head on the other end of the extension for receiving an endplate perpendicular to the axis;
a locating formation on each endplate receiving head for locating the endplate at a specific angle about the axis;
a selection of one or more end plates receivable on the endplate receiving head with the tissue engaging surface of the endplates being transverse to the axis of the spacer at differing angles; and
wherein the locating formation on each endplate receiving head for locating the endplate at a specific angle about the axis is a notch formed in the head or endplate with a complementary stub formation which fits snugly into the notch formed on either the endplate or head, as the case may be.

2. A spacer as claimed in claim 1, wherein the cylindrical extension is provided with one or more lateral threaded sockets extending into the connector transverse to the axis.

3. An inter-body spacer introducer or implantation tool, which introducer comprises:
a thread tipped rod which engages, in use, one of the lateral threaded sockets of the connector;
a sleeve over the rod, which rod and sleeve can be rotated relative to each other and which sleeve at the threaded end of the rod is provided with gears for engaging, in use, the gears of the pinion to rotate the pinion, in turn, to adjust the length of the inter-body spacer;
an outer tube covering the rod and sleeve with a handle at the one end and locating means for locating the inter-body spacer at the other end with the thread tipped rod engaged as well as the gears of the sleeve engaged, with the locating means being in the form of U-shaped arms which fit snugly and complementally over the connector to locate the inter-body spacer and the introducer square to each other; and wherein
the end of the sleeve opposite the gears extends past the handle of the outer tube and fitted with its own handle; and wherein
a window is provided in the sleeve, between the handle of the outer tube and the handle of the sleeve to provide access to rod, which rod is provided with gripping means which can be gripped through the window to allow the rod to be rotated relative to the sleeve and outer tube.

4. An elongate thoracolumbar lateral plate, which can be used to complement the inter body spacer as claimed in claim 1, which lateral plate comprises:
two parts of which one has a slot and the other a lockable screw, which screw is receivable in the slot and the two parts are slideably connected, and wherein the opposed end of each part is provided with a pair of first countersunk holes for receiving surgical screws there through, and wherein a second countersunk hole is provided adjacent each first countersunk hole, intersecting therewith.
